(19) United States
(12) Patent Application Publication
(10) Pub. No.: US 2022/0000744 A1
(43) Pub. Date: Jan. 6, 2022

(54) ORGANIC SILICON COMPOUNDS IN ANHYDROUS PHASE FOR INCREASING THEIR STORAGE STABILITY

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/288,864

(22) PCT Filed: Oct. 31, 2019
(86) PCT No.: PCT/EP2019/079776

§ 371 Date: Apr. 26, 2021

(30) Foreign Application Priority Data
Oct. 31, 2018 (DE) 10 2018 127 290.1

(51) Int. Cl. A61K 8/58; A61K 8/31; A61K 8/34; A61Q 5/00

(52) U.S. Cl. CPC A61K 8/585; A61K 8/31; A61K 2800/52; A61Q 5/00; A61K 2800/31; A61K 8/342

(57) ABSTRACT

The present disclosure relates to an anhydrous carrier medium for increasing the storage stabilization of organic silicon compounds comprising at least one organic silicon compound, at least one branched or linear C8-C30 alkane, at least one branched or linear C10-C30 fatty alcohol, and at least one branched or linear C2-C8 monohydric alcohol. The present disclosure further relates to the use of the anhydrous carrier medium for the production of cosmetic compositions.

### CROSS-REFERENCE TO RELATED APPLICATION

This application is a U.S. National-Stage entry under 35 U.S.C. § 371 based on International Application No. PCT/EP2019/079776, filed Oct. 31, 2019, which was published under PCT Article 21(2) and which claims priority to German Application No. 10 2018127290.1, filed Oct. 31, 2018, which are all hereby incorporated in their entirety by reference.

### TECHNICAL FIELD

The present disclosure relates to an anhydrous carrier medium for increasing the storage stability of organic silicon compounds, wherein the anhydrous carrier medium comprises an organic silicon compound and a branched or linear C8-C30 alkane.

### BACKGROUND

The exposure of hair to external chemicals from a variety of different sources poses challenges for the development of cosmetic care products. Air and water impurities have a detrimental effect on skin and hair. The action of various atmospheric pollutants can be intensified in the presence of other atmospheric pollutants and under UV radiation action.

It is known that the toxicity of gaseous pollutants of air, such as sulfur dioxide, ozone and nitrogen oxides, is related in particular to their initiator activity for free radicals which cause damage to living organisms. Free radicals are metabolites that also occur naturally in the body. In this case, one speaks of “oxidative da mage” Free radicals can also cause visible hair damage, for example, as a reduction in shine as well as grip and/or fading of hair color.

Furthermore, often changing consumer demands for a certain hair texture are associated with recurring hair chemical stress. For example, hair colorings stress the hair, as a result of which special, intensive care may be necessary.

For the protection and care of the hair, organosilicon compounds from the group of silanes which comprise at least one hydroxyl group and/or hydrolyzable group are described in the prior art. Due to the presence of hydroxy groups and/or hydrolyzable groups, silanes are reactive substances that hydrolyze or oligomerize or polymerize in the presence of water. The oligomerization or polymerization of the silanes initiated by the presence of the water, when applied to a keratinous material, ultimately leads to the formation of a film that can provide a protective effect.

### BRIEF SUMMARY

Anhydrous carrier mediums for the storage stabilization of organic silicon compounds are provided. In an exemplary embodiment, an anhydrous carrier medium for the storage stabilization of organic silicon compounds includes at least one organic silicon compound, at least one branched or linear C8-C30 alkane, at least one branched or linear C10-C30 fatty alcohol, and at least one branched or linear C2-C8 monohydric alcohol.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The problem with the use of organosilicon compounds is their instability to water. Aqueous systems for hair treatment are thus disadvantageous if they contain organic silicon compounds as active substances.

The problem underlying the present disclosure is to provide a medium that increases the storage stability of organic silicon compounds. In particular, the present disclosure was directed to providing an anhydrous carrier medium that stabilizes organic silicon compounds to be ready for use in the preparation of a ready-to-use cosmetic composition.

This task is solved by an anhydrous carrier medium comprising at least one organic silicon compound, at least one branched or linear C8-C30 alkane, at least one branched or linear C10-C30 fatty alcohol, and at least one branched or linear monohydric C2-C8 alcohol.

The carrier medium is used to provide an organosilicon compound. In the carrier medium, the organic silicon compound should remain lab-stable. The carrier medium is then used in combination with one or more other compositions containing active ingredients for hair care, which may be water-based. The fact that the carrier medium can then be combined with one or the other compositions just before use means that the active ingredient organosilicon compound remains stable until use.

In the context of the current invention, the term “water-free” should preferably be understood to mean that water is not added to the carrier medium or that the carrier medium is not water-based. More preferably, the water content of the anhydrous carrier medium is less than about 5% by weight, even more preferably less than about 2% by weight, most preferably less than about 1% by weight, based on the total weight of the anhydrous carrier medium. When small amounts of moisture are present, a small portion of the organic silicon compound can hydrolyze and the hydrolysate is present in equilibrium with free water. This amount of water is preferably present in the abovementioned amounts.

Keratinous material includes hair, skin, nails (such as fingernails and/or toenails). Wool, furs and feathers also fall under the definition of keratinous material.

Preferably, keratinous material is understood to be human hair, human skin and nails, especially fingernails and toenails. In particular, keratinous material is understood to mean human hair, especially head and/or beard hair.

As a first constituent essential to the invention, the anhydrous carrier medium contains at least one organic silicon compound, namely the one or more compounds which are to be stabilized, in order to increase the storage stability of organic silicon compounds. Preferred organic silicon compounds are selected from silanes having one, two or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The anhydrous support medium for increasing storage stability contains at least one organic silicon compound preferably selected from silanes having one, two or three silicon atoms, wherein the organic silicon compound comprises one or more hydroxyl groups or hydrolyzable groups per molecule.

In a particularly preferred method, the anhydrous carrier medium has at least one organic silicon compound selected from silanes having one, two or three silicon atoms in order to increase storage stability, where the organic silicon compound also comprises one or more basic groups and one or more hydroxyl groups or hydrolysable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a Di(C₁-C₆)alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the anhydrous carrier medium for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another particularly preferred embodiment, the anhydrous carrier medium comprises at least one organic silicon compound of formula (I) and/or (II),

R₁R₂N-L-Si(OR₃)ₐ(R₄)_{b} (I),
where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group, and
a stands for the number 3 and
b stands for the number 0.

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
where
R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)—Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. More preferably -L- stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. In particular, R₃ and R₄ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the anhydrous carrier medium contains at least one organic silicon compound of formula (I) or formula (II) in which the radicals R₃, R₄ independently represent a methyl group or an ethyl group.

Particularly well-suited organic silicon compounds of formula (I) are

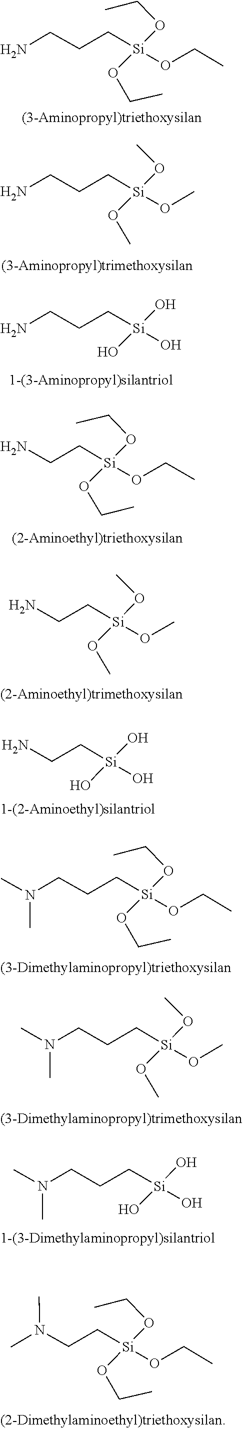

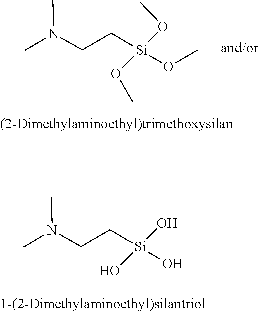

The aforementioned organic silicon compound of formula (I) is commercially available.

(3-aminopropyl)trimethoxysilane, for example, can be purchased from Sigma-Aldrich. (3-Aminopropyl)triethoxysilane is also commercially available from Sigma-Aldrich.

In another form, the anhydrous carrier medium comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

The organosilicon compounds of formula (II) as contemplated herein each carry the silicon-containing groups (R₅O)_{c}(R₆)_{d}Si— and —Si(R₆′)_{d′}(OR_{5′})_{c} at both ends.

In the central part of the molecule of formula (II) there are the groups -(A)_{c}- and —[NR₇-(A′)_{f}]—

and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group including -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Highly preferred carrier media contain an organic silicon compound in which the radicals c and c′ both stand for the number 3. In this case d and d′ both stand for the number 0.

In another preferable form, the anhydrous carrier medium comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and -[NR7-(A′)]_{f}- and —[O-(A″)]_{g}- and -[NR8-(A′″)]ₕ- are located in the middle part of the organic silicon compound of formula (II).

In this context, the presence of certain groups has proved to be particularly advantageous with regard to increasing the conditioning effect. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]—Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping -[NR7-(A′)]-.

When the radical h represents the number 1, the organic silicon compound of formula (II) contains a structural grouping -[NR8-(A′″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)—Si(R₆″)_{d}″(OR₅″)_{c}″ (III),

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound as contemplated herein contains the grouping [NR₇-(A′)] but not the grouping —[NR₈-(A′″)]. If the radical R7 now stands for a grouping of the formula (III), the agent (a) contains an organic silicone compound with 3 reactive silane groups.

In another preferable form, the anhydrous carrier medium comprises at least one organic silicon compound of formula (II)

(R₅O)_{c}(R₆)_{d}Si-(A)_{c}-[NR₇-(A′)]_{f}—[O-(A″)]_{g}—[NR₈-(A′″)]ₕ—Si(R₆′)_{d′}(OR₅′)_{c′} (II),

where
e and f both stand for the number 1,
g and h both stand for the number 0,
A and A′ independently of one another represent a linear, divalent C1-C6 alkylene group and
R₇ represents a hydrogen atom, a methyl group, a 2-hydroxyethyl group, a 2-alkenyl group, a 2-aminoethyl group or a group of formula (III).

In another preferable form, the anhydrous carrier medium comprises at least one organic silicon compound of formula (II), wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R₇ represents a hydrogen atom, a methyl group, a 2-hydroxyethyl group, a 2-alkenyl group, a 2-aminoethyl group or a group of formula (III).

Organic silicon compounds of formula (II) that are suitable for solving the problem are

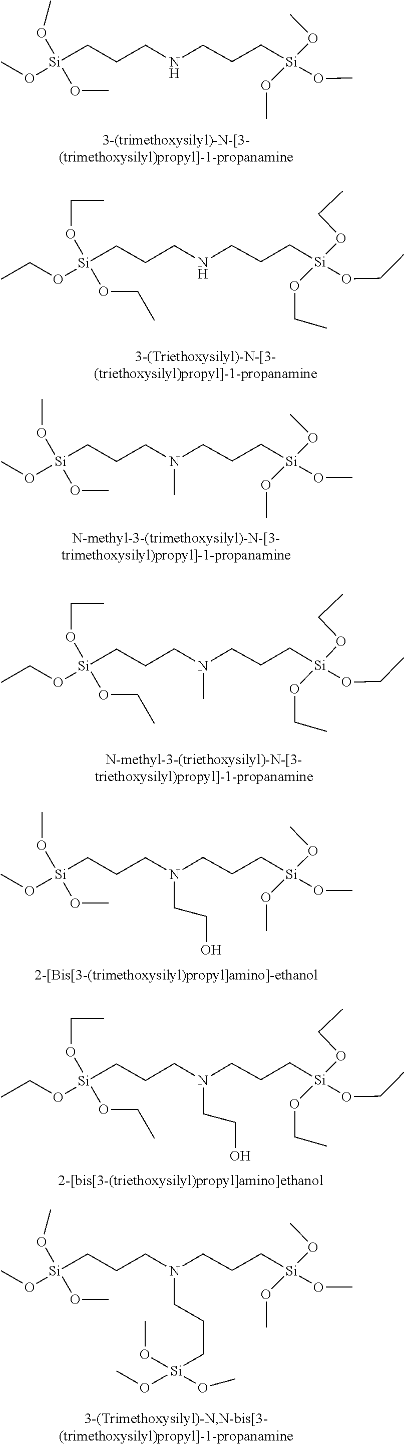

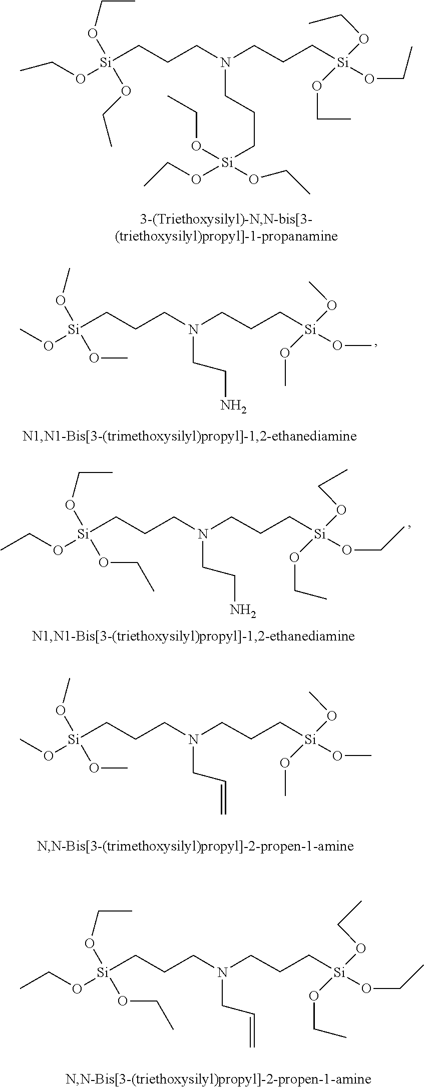

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, having the CAS number 13497-18-2, can be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous if the agent for treating a keratinous material applied to the hair contains at least one organic silicon compound of formula (IV)

R₉Si(OR₁₀)ₖ(R₁₁)ₘ (IV).

Consequently, the anhydrous carrier medium also contains an organic silicon compound of formula (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further preferred embodiment, the anhydrous carrier medium contains, in addition to the organic silicon compound or compounds of the formula (I), at least one further organic silicon compound of the formula (IV)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a likewise preferred embodiment, the anhydrous carrier medium contains, in addition to the organic silicon compound or compounds of the formula (I), at least one further organic silicon compound of the formula (IV)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further preferred embodiment, the anhydrous carrier medium contains, in addition to the organic silicon compounds of the formula (I) and (II), at least one further organic silicon compound of the formula (IV)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a hydrogen atom or a C₁-C₆ alkyl group. It is particularly preferred that R₁₀ represents a methyl group or an ethyl group.

In the organic silicon compounds of formula (IV), the radical R₁₁ represents a C₁-C₆ alkyl group. It is particularly preferred that R₁₁ represents a methyl group or an ethyl group.

Furthermore k stands for a whole number from 1 to 3, and m stands for the whole number 3-k. If k stands for the number 3, then m is equal to 0. If k stands for the number 2, then m is equal to 1. If k stands for the number 1, then m is equal to 2.

It has proved very advantageous for the anhydrous carrier medium to contain at least one organic silicon compound of the formula (IV) in which the radical k represents the number 3. In this case the rest m stands for the number 0.

Organic silicon compounds of the formula (IV) that are particularly suitable for solving the problem are

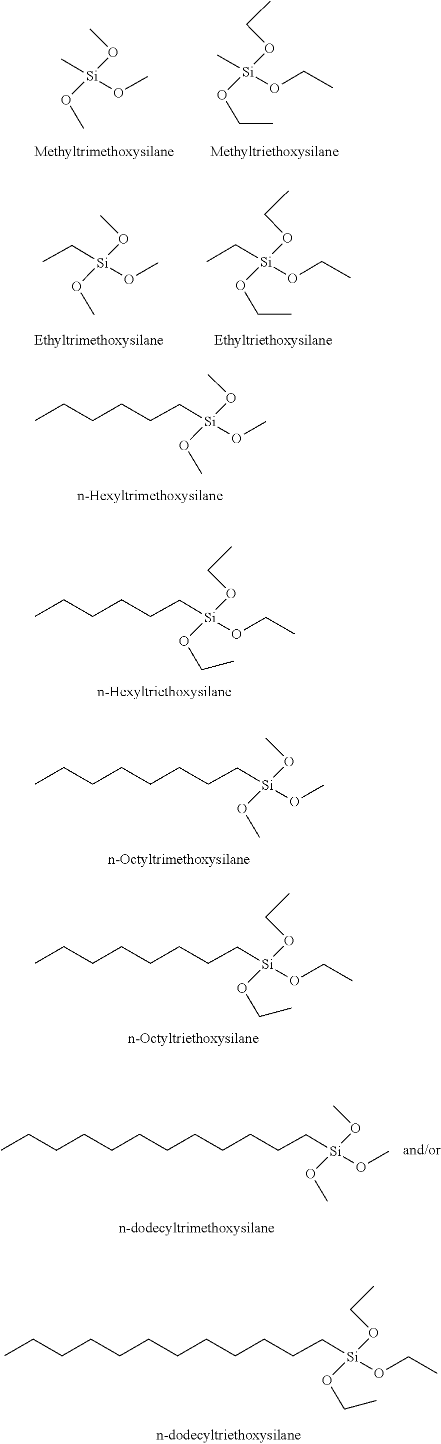

and also propyltrimethoxysilane, propyltriethoxysilane, octadecyltrimethoxysilane and/or octadecyltriethoxysilane.

The organic silicon compounds described above are reactive compounds.

It was found that particularly stable and uniform films could be obtained on the keratinous material even when the anhydrous carrier medium contained two structurally different organic silicon compounds.

In a preferred embodiment, an anhydrous carrier medium comprises at least one organic silicone compound of formula (I) and at least one organic silicone compound of formula (IV).

In an explicitly very particularly preferred embodiment, an anhydrous carrier medium exemplified contains at least one organic silicon compound of the formula (I) which is selected from the group including (3-aminopropyl)triethoxysilane and (3-aminopropyl)trimethoxysilane, and additionally contains at least one organic silicon compound of the formula (IV) which is selected from the group including methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an anhydrous carrier medium is exemplified in that the anhydrous carrier medium comprises—based on the total weight of the anhydrous carrier medium:
about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
about 3.2 to about 10% by weight of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in the case of organic silicon compounds having at least one hydrolyzable group. The hydrolysis products and/or organic silicon compounds having at least one hydroxyl group can react with one another in a condensation reaction. For this reason, both the organosilicon compounds having at least one hydrolyzable group and their hydrolysis and/or condensation products may be present in the anhydrous carrier medium. When organosilicon compounds having at least one hydroxyl group are used, both the organic silicon compounds having at least one hydroxyl group and their condensation products can be present in the anhydrous carrier medium.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium of monomeric organic silicon compounds shifts to condensation product.

In the context of the present invention, FIGURES in wt.-% are—unless stated otherwise—always based on the total weight of the anhydrous carrier medium.

As a second component essential to the invention, the anhydrous carrier medium contains a branched or linear alkane. In the course of the work leading to the present invention, it has been found that, in order to achieve a particularly good care effect, it is particularly advantageous if the organic silicon compound, for example (3-aminopropyl)triethoxysilane, is combined with a branched or linear C8-C30 alkane. By excluding water in the anhydrous carrier medium, the organic silicon compound is protected from premature hydrolysis and is converted into a hair care emulsion only when required by mixing with a water phase, which activates the organic silicon compound by employing a hydrolysis reaction only shortly before application. Surprisingly, it was found that especially the combination of (3-aminopropyl)triethoxysilane with C8-C30 alkanes increased the cosmetic acceptability. The hair is soft, manageability is significantly increased, and the hair surface is more hydrophobic, especially in chemically treated hair.

According to preferred embodiments of the present invention, the alkane is a C10-C24 alkane, more preferably a C12-C18 alkane, and even more preferably a C14-C16 alkane. According to another preferred embodiment, the alkane is present in the anhydrous carrier medium in an amount of from about 1 to about 50% by weight, preferably from about 2 to about 45% by weight, more preferably from about 3 to about 40% by weight, even more preferably from about 4 to about 35% by weight, based on the total weight of the anhydrous carrier medium.

The combination of the organosilicon compound, especially (3-aminopropyl)triethoxysilane, with the alkane forms a layer on the hair. The hair surface is hydrophobized again with oxidatively damaged hair, which leads to the reduction of frizz. In addition, the manageability of the hair is improved.

As a third constituent essential to the invention, the anhydrous carrier medium contains at least one branched or linear C10-C30 fatty alcohol. This formulation always means that the hydrocarbon chain has 10 to 30 carbon atoms.

According to a preferred embodiment of the present invention, the fatty alcohol is a C12-C24 fatty alcohol, preferably a C14-C18 fatty alcohol. According to another preferred embodiment of the present invention, the fatty alcohol is present in the anhydrous carrier medium in an amount of about 5 to about 50% by weight, preferably from about 6 to about 45% by weight, more preferably from about 7 to about 40% by weight, even more preferably from about 8 to about 35% by weight, based on the total weight of the anhydrous carrier medium.

As a fourth ingredient essential to the invention, the anhydrous carrier medium contains at least one branched or linear C2-C8 monohydric alcohol. According to a preferred embodiment of the present invention, the alcohol is a C3-C6 alcohol, preferably a C4-C5 alcohol. According to another preferred embodiment, the alcohol is present in the anhydrous carrier medium in an amount of from about 4 to about 50% by weight, preferably from about 6 to about 45% by weight, more preferably from about 8 to about 40% by weight, even more preferably from about 10 to about 30% by weight, based on the total weight of the anhydrous carrier medium. A monohydric alcohol is to be understood as meaning an alcohol having only one OH function.

The present disclosure also relates to a cosmetic composition. This comprises the anhydrous carrier medium according to the present invention, which is combined with an aqueous phase or alternatively also with a further anhydrous phase. In this way, cosmetic compositions can be provided which contain all desired components which are advantageous for the cosmetic composition, the organic silicon compound being protected from hydrolysis.

A preferred embodiment of the present disclosure relates to a cosmetic composition comprising an anhydrous carrier medium according to the present disclosure and an aqueous phase, wherein the anhydrous carrier medium or the aqueous phase comprises a cationic surfactant, preferably an alkyltrimonium compound having one or more C8-C22, more preferably C10-C18, still more preferably C12-C16 alkyl groups, or wherein the cationic surfactant is one of the following formula,

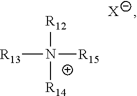

wherein
R₁₂, R₁₃, R₁₄ independently represent a C1-C6 alkyl group, a C2-C6 alkenyl group or a C2-C6 hydroxyalkyl group,
R₁₅ represents a C8-C28 alkyl group, preferably a C10-C22 alkyl group, and
X— stands for a physiologically compatible anion,
or wherein the cationic surfactant is one of the following formula,

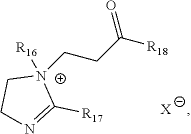

wherein
R₁₆ stands for a C1-C6 alkyl group
R₁₇, R₁₈ are independently a C7-C27 alkyl group, preferably a C10-C22 alkyl group, and
X— stands for a physiologically compatible anion,
or wherein the cationic surfactant is one of the following formula,

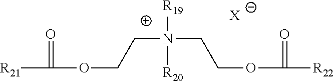

wherein
R₁₉, R₂₀ independently represent a C1-C6 alkyl group, a C2-C6 alkenyl group or a C2-C6 hydroxyalkyl group,
R₂₁, R₂₂ are independently a C7-C27 alkyl group, preferably a C10-C22 alkyl group, and
X— stands for a physiologically compatible anion,
or wherein the cationic surfactant is one of the following formula,

NR₂₃R₂₄R₂₅,

wherein
R₂₃, R₂₄ independently represent a C1-C6 alkyl group, a C2-C6 alkenyl group or a C2-C6 hydroxyalkyl group, and
R₂₅ represents a C8-C28 alkyl group, preferably a C10-C22 alkyl group.

In the current invention, the surfactants serve to emulsify the aqueous phase and the carrier medium, or, more generally, to emulsify the water phase and the oil phase.

According to a preferred embodiment of the present invention, the cationic surfactant is present in aqueous phase in an amount of from about 0.1 to about 5 wt-%, preferably from about 0.5 to about 4 wt-%, more preferably from about 1 to about 3 wt-%, based on the total weight of the aqueous phase.

A preferred embodiment of the present disclosure relates to a cosmetic composition in which the anhydrous carrier medium or aqueous phase comprises a nonionic surfactant, wherein the nonionic surfactant is preferably a fatty alcohol ethoxylate in which the fatty alcohol portion of the fatty alcohol ethoxylate has an alkyl chain length of C4-C30, preferably C6-C25, more preferably C8-C20, and/or in which the number of ethoxy groups in the fatty alcohol ethoxylate is from about 2 to about 120, preferably from about 4 to about 100, more preferably from about 6 to about 80, still more preferably from about 8 to about 60, most preferably from about 10 to about 40, and/or wherein the nonionic surfactant is present in an amount of from about 0.1 to about 10% by weight, preferably from about 0.25 to about 7.5% by weight, more preferably from about 0.5 to about 5% by weight, based on the total weight of the anhydrous carrier medium.

Another preferred embodiment of the present disclosure relates to a cosmetic composition wherein the anhydrous carrier medium or aqueous phase comprises an anionic surfactant, wherein the anionic surfactant is preferably a fatty alcohol sulfate having a chain length of C8-C22, preferably of C10-C20, more preferably of C12-C18, and/or wherein the anionic surfactant is present in an amount of from about 0.1 to about 10% by weight, preferably from about 0.25 to about 7.5% by weight, more preferably from about 0.5 to about 5% by weight, based on the total weight of the anhydrous carrier medium, in the anhydrous carrier medium.

According to another preferred embodiment of the present invention, the weight ratio of the anhydrous carrier medium to the aqueous phase is in the range of about 1 to about 10 to about 10 to about 1, preferably from about 5 to about 1 to about 1 to about 5, more preferably from about 2 to about 1 to about 1 to about 2.

Further ingredients of the hair treatment compositions are described below which, in addition to the mandatory ingredients described above, may be present in the anhydrous carrier medium or the aqueous phase.

According to further preferred embodiments, the anhydrous carrier medium or the aqueous phase further comprises a skin moisturizing or further skin care agent selected from the group including glycerol, urea, hyaluronic acid, silanol esters of hyaluronic acid, panthenol, taurine, ceramides, phytosterols, aloe vera extracts, creatine, creatinine, sodium hyaluronate, polysaccharides, biosaccharides gum-1, cucumber extracts, butylene glycol, propylene glycol, methyl propanediol, ethylhexylglycerol, sorbitol, amino acids, glycine, glycine soy, histidine, tyrosine or tryptophan being particularly preferred amino acids, amino acid derivatives, natural betaine compounds, pyrrolidonecarboxylic acid or a salt of pyrrolidonecarboxylic acid, lactic acid, lactates, in particular sodium lactate, and/or ethylhexyloxyglycerol. In particular, the selection of these skin moisturizers increases the nourishing character of the anhydrous carrier medium.

According to another preferred embodiment, the anhydrous carrier medium or the aqueous phase contains several surfactants. It is particularly preferred that the anhydrous carrier medium or the aqueous phase contains two structurally different surfactants, preferably the anhydrous carrier medium or the aqueous phase contains two structurally different cationic surfactants, two structurally different anionic surfactants, a cationic surfactant and a nonionic surfactant, or an anionic surfactant and a nonionic surfactant.

According to a preferred embodiment of the present invention, the cationic surfactant used comprises a hydrophobic head group with a cationic charge and one or two hydrophobic end portions, wherein the hydrophobic end portion(s) represent straight-chain or branched, saturated or mono- or polyunsaturated alkyl groups, preferably having a chain length of C6 to C30, more preferably C8 to C26, particularly preferably C10 to C22. According to another preferred embodiment, the cationic surfactant has an ester function, an ether function, a ketone function, an alcohol function, or an amide function.

The cationic surfactants of formula NR₂₃R₂₄R₂₅ are amine derivatives, so-called pseudoquats. The organic radicals R₂₃, R₂₄ and R₂₅ are bonded directly to the nitrogen atom. In the acidic pH range, these are cationized, i.e. the nitrogen atom is then protonated. The physiologically compatible counterions are suitable as counterions. Steamidopropyl dimethylamine is particularly preferred among these cationic surfactants.

In principle, all physiologically compatible counterions can be used as counterions of the present compounds, which are present as salts.

According to a preferred embodiment of the present invention, the anhydrous carrier medium or aqueous phase contains as a further component a further nonionic surfactant, which preferably comprises a nonionic surfactant selected from the group including the following:
Alkyl glucamide comprising a saturated or unsaturated, branched or unbranched C₆ to C₂₂, preferably C₁₀ to C₁₈, more preferably C₁₂ to C₁₆ alkyl group,
An alkyl fructoside comprising a saturated or unsaturated, branched or unbranched C₆ to C₂₂, preferably C₁₀ to C₁₈, more preferably C₁₂ to C₁₆ alkyl group,
alkyl glucoside comprising a saturated or unsaturated, branched or unbranched C₆ to C₂₂, preferably C₁₀ to C₁₈, more preferably C₁₂ to C₁₆ alkyl group, and
Alkyl alcohol alkoxylate of the formula R₁₀(OR₁₁)ₘOH, in which R₁₀ represents a linear or branched C₆ to C₂₂, preferably C₁₀ to C₁₈, more preferably C₁₂ to C₁₆ alkyl group, R₁₁ represents a C₂ to C₄, preferably a C₂ alkyl group, and ₘ represents 1 to 10, preferably 2 to 6, more preferably 2 to 6.

According to preferred embodiments of the present invention, one or more further anionic surfactants are present as a component in the anhydrous carrier medium or aqueous phase, which is preferably selected from the group including
straight-chain or branched, saturated or mono- or polyunsaturated alkylsulfonates having 8 to 24, preferably 12 to 22, more preferably 16 to 18, carbon atoms,
linear alpha-olefin sulfonates having 8 to 24, preferably 12 to 22, more preferably 16 to 18, carbon atoms,
Alkyl sulfates and alkyl polyglycol ether sulfates of the formula R₉—O—(CH₂—CH₂O)n-SO₃X, in which R₉ is preferably a straight-chain or branched, saturated or mono- or polyunsaturated alkyl or alkenyl radical having 8 to 24, preferably 12 to 22, more preferably 16 to 18 carbon atoms, n is 0 or 1 to 12, more preferably 2 to 4, and X is an alkali metal or alkaline earth metal ion or protonated triethanolamine or the ammonium ion,
straight-chain or branched, saturated or mono- or polyunsaturated alkylcarboxylic acids containing 8 to 24, preferably 12 to 22, more preferably 16 to 18 carbon atoms,
straight-chain or branched, saturated or mono- or polyunsaturated alkyl phosphates containing 8 to 24, preferably 12 to 22, more preferably 16 to 18 carbon atoms,
Alkyl isethionate whose alkyl group is selected from a branched or unbranched C₆ to C₂₂, preferably C₁₀ to C₁₈, more preferably C₁₂ to C₁₆ alkyl group, in particular sodium cocoyl isethionate,
Alkyl glycoside carboxylic acids whose alkyl group is selected from a branched or unbranched C₆ to C₂₂, preferably C₁₀ to C₁₈, more preferably C₁₂ to C₁₆ alkyl group,
Alkyl sulfosuccinates, the two alkyl groups of which are selected from identical or different, branched or unbranched C₂ to C₁₂, preferably C₄ to C₁₀, more preferably C₆ to C₈ alkyl groups,
Alkyl taurates, whose alkyl group is selected from a branched or unbranched C₆ to C₂₂, preferably C₁₀ to C₁₈, more preferably C₁₂ to C₁₆ alkyl group,
Alkyl sarcosinates, whose alkyl group is selected from a branched or unbranched C₆ to C₂₂, preferably C₁₀ to C₁₈, more preferably C₁₂ to C₁₆ alkyl group,
Sulfonates of unsaturated fatty acids with 8 to 24, preferably 12 to 22, more preferably 16 to 18 C atoms and 1 to 6 double bonds,
   wherein the counterion of the anionic surfactant is an alkali or alkaline earth metal ion or a protonated triethanolamine or the ammonium ion.

Very preferably, aqueous phase contains a surfactant mixture of anionic and amphoteric/zwitterionic surfactants sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO— or —SO₃⁻ group in the molecule. Amphoteric/zwitterionic surfactants also include surface-active compounds which, in addition to a C8-C24 alkyl or acyl group, contain at least one free amino group and at least one —COOH or —SO₃H group and are capable of forming internal salts.

According to a preferred embodiment of the present invention, the anhydrous carrier medium or the aqueous phase contains at least one amphoteric surfactant as a further component. Preferably, the amphoteric surfactants in the anhydrous carrier medium are selected from the group including
Alkyl betaine comprising at least one saturated or unsaturated, branched or unbranched C₆ to C₂₂, preferably C₁₀ to C₁₈, more preferably C₁₂ to C₁₆ alkyl group,
alkyl amphodiacetate or alkyl amphodiacetate comprising a saturated or unsaturated, branched or unbranched C₆ to C₂₂, preferably C₁₀ to C₁₈, more preferably C₁₂ to C₁₆ alkyl group, with an alkali or alkaline earth metal counterion, and
alkylamidopropyl betaine comprising at least one saturated or unsaturated, branched or unbranched C₆ to C₂₂, preferably C₁₀ to C₁₈, more preferably C₁₂ to C₁₆ alkyl group.

Particularly suitable amphoteric/zwitterionic surfactants include those known under the INCI designation cocamidopropyl betaine and disodium cocoamphodiacetate.

The anhydrous support medium for stabilizing organic silicon compounds may in particular be used to prepare an agent for purifying a keratinous material, an agent for caring for a keratinous material, an agent for caring for and purifying a keratinous material, an agent for coloring a keratinous material, and/or an agent for temporarily reshaping a keratinous material.

It may be preferred that the anhydrous carrier medium or aqueous phase for treating a keratinous material further comprises from about 0.001 to about 20 wt % of at least one quaternary compound. This applies in particular to anhydrous carrier media or aqueous phases used for the preparation of an agent for the care of a keratinous material or for the care and cleaning of a keratinous material.

It is preferred that the at least one quaternary compound is selected from at least one of the groups including

i) the monoalkylquats and/or
ii) the esterquats and/or
(iii) the quaternary imidazolines of formula (Tkat2),

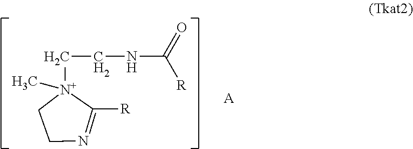

in which the radicals R independently of one another each represent a saturated or unsaturated, linear or branched hydrocarbon radical having a chain length of 8 to 30 carbon atoms and A represents a physiologically tolerated anion, and/or
iv) of the amidoamines and/or cationized amidoamines and/or
v) Poly(methacryloyloxyethyltrimethylammonium compounds) and/or;
vi) quaternized cellulose derivatives, in particular polyquaternium 10, polyquaternium-24, polyquaternium-27, polyquaternium-67, polyquaternium-72, and/or
vii) cationic alkyl polyglycosides and/or
viii) cationized honey and/or
ix) cationic guar derivatives and/or
x) chitosan and/or
xi) polymeric dimethyldiallylammonium salts and copolymers thereof with esters and amides of acrylic acid and methacrylic acid, in particular polyquaternium-7 and/or
xii) copolymers of vinylpyrrolidone with quaternized derivatives of dialkylaminoalkyl acrylate and methacrylate, especially polyquaternium-11 and/or
xiii) vinylpyrrolidone-vinylimidazolium methochloride copolymers, in particular polyquaternium-16 and/or
xiv) quaternized polyvinyl alcohol and/or
xv) polyquaternium-74
and mixtures thereof.

In particular, it is preferred that the anhydrous carrier medium or aqueous phase contains a cationic homopolymer falling under the INCI designation polyquaternium-37 as quaternary compounds.

It may be preferred that the anhydrous carrier medium or aqueous phase further comprises a firming compound, preferably selected from the group including waxes, synthetic polymers and mixtures thereof.

In order to meet the different requirements of cosmetic compositions which are used for the preparation of an agent for the treatment of a keratinous material for the temporary reshaping of a keratinous materials (=styling agent), a large number of synthetic polymers have already been developed as strengthening compounds which can be used in the agent for the treatment of a keratinous material. Alternatively or additionally, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic and amphoteric strengthening polymers.

Suitable synthetic polymers include, for example, polymers with the following INCI names: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Homopolyacrylic acid (INCI: Carbomer), which is commercially available under the name Carbopol® in various forms, is suitable as a firming compound.

Preferably, the fixing compound a polymer containing vinylpyrrolidone. Particularly preferably, the firming compound comprises a polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel.

Accordingly, it is particularly preferred that the firming compound comprises a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

In addition to or as an alternative to a synthetic polymer, the cosmetic compositions may contain at least one natural or synthetic wax which has a melting point of above 37° C. as a fixing compound.

Natural or synthetic waxes may be solid kerosenes or isoparaffins, vegetable waxes such as candelilla wax, camauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified and synthetic waxes can be used alone or in combination. However, this is not to include alkanes, which are necessarily contained in the anhydrous carrier medium as contemplated herein. Several waxes can also be used. Furthermore, a number of wax compounds, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

Preferably, the wax is selected from carnauba wax (INCI: Copernicia Cerifera Cera) Beeswax (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and Petrolatum.

Wax or the wax components should be solid at 25° C. and should melt in the range >37° C.

The anhydrous carrier medium or aqueous phase preferably contains the firming compound in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic polymers, anionic polymers, (further) cationic polymers, waxes, protein hydrolysates, amino acids, oligopetides, vitamins, provitamins, vitamin precursors, betaines, biochinones, purine (derivatives), plant extracts, silicones, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In preferred embodiments 1 to 48, the preferred organic silicon compounds are combined with the preferred alkanes and alcohols in an anhydrous carrier medium as contemplated herein.

| | Silane compound | other ingredients |
|---|---|---|
| 1 | (3-Aminopropyl)trimethoxysilane | branched or linear C14-C16 alkane + |
| | | branched or linear C14-C18 fatty |
| | | alcohol + branched or linear |
| | | monohydric C4-C5 alcohol |
| 2 | (3-Aminopropyl)triethoxysilan | branched or linear C14-C16 alkane + |
| | | branched or linear C14-C18 fatty |
| | | alcohol + branched or linear |
| | | monohydric C4-C5 alcohol |
| 3 | (2-Aminoethyl)trimethoxysilane | branched or linear C14-C16 alkane + |
| | | branched or linear C14-C18 fatty |
| | | alcohol + branched or linear |
| | | monohydric C4-C5 alcohol |
| 4 | (2-Aminoethyl)triethoxysilane | branched or linear C14-C16 alkane + |
| | | branched or linear C14-C18 fatty |
| | | alcohol + branched or linear |
| | | monohydric C4-C5 alcohol |
| 5 | (3-Dimethylaminopropyl)trimethoxysilane | branched or linear C14-C16 alkane + |
| | | branched or linear C14-C18 fatty |
| | | alcohol + branched or linear |
| | | monohydric C4-C5 alcohol |
| 6 | (3-Dimethylaminopropyl)triethoxysilane | branched or linear C14-C16 alkane + |
| | | branched or linear C14-C18 fatty |
| | | alcohol + branched or linear |
| | | monohydric C4-C5 alcohol |
| 7 | (2-Dimethylaminoethyl)trimethoxysilane | branched or linear C14-C16 alkane + |
| | | branched or linear C14-C18 fatty |
| | | alcohol + branched or linear |
| | | monohydric C4-C5 alcohol |
| 8 | (2-Dimethylaminoethyl)triethoxysilane | branched or linear C14-C16 alkane + |
| | | branched or linear C14-C18 fatty |
| | | alcohol + branched or linear |
| | | monohydric C4-C5 alcohol |
| 9 | 3-(trimethoxysilyl)-N-[3-(trimethoxysilyl)propyl]-1-propanamine | branched or linear C14-C16 alkane + |
| | | branched or linear C14-C18 fatty |
| | | alcohol + branched or linear |
| | | monohydric C4-C5 alcohol |
| 10 | 3-(triethoxysilyl)-N-[3-(triethoxysilyl)propyl]-1-propanamine | branched or linear C14-C16 alkane + |
| | | branched or linear C14-C18 fatty |
| | | alcohol + branched or linear |
| | | monohydric C4-C5 alcohol |
| 11 | N-methyl-3-(trimethoxysilyl)-N-[3- | branched or linear C14-C16 alkane + |
| | (trimethoxysilyl)propyl]-1-propanamine | branched or linear C14-C18 fatty |
| | | alcohol + branched or linear |
| | | monohydric C4-C5 alcohol |
| 12 | N-Methyl-3-(triethoxysilyl)-N-[3- | branched or linear C14-C16 alkane + |
| | (triethoxysilyl)propyl]-1-propanamine | branched or linear C14-C18 fatty |
| | | alcohol + branched or linear |
| | | monohydric C4-C5 alcohol |
| 13 | 2-[Bis[3-(trimethoxysilyl)propyl]amino]- | branched or linear C14-C16 alkane + |
| | ethanol | branched or linear C14-C18 fatty |
| | | alcohol + branched or linear |
| | | monohydric C4-C5 alcohol |
| 14 | 2-[bis[3-(triethoxysilyl)propyl]amino]ethanol | branched or linear C14-C16 alkane + |
| | | branched or linear C14-C18 fatty |
| | | alcohol + branched or linear |
| | | monohydric C4-C5 alcohol |
| 15 | 3-(trimethoxysilyl)-N,N-bis[3- | branched or linear C14-C16 alkane + |
| | (trimethoxysilyl)propyl]-1-propanamine | branched or linear C14-C18 fatty |
| | | alcohol + branched or linear |
| | | monohydric C4-C5 alcohol |
| 16 | 3-(triethoxysilyl)-N,N-bis[3- | branched or linear C14-C16 alkane + |
| | (triethoxysilyl)propyl]-1-propanamine | branched or linear C14-C18 fatty |
| | | alcohol + branched or linear |
| | | monohydric C4-C5 alcohol |
| 17 | N1,N1-Bis[3-(trimethoxysilyl)propyl]-1,2- | branched or linear C14-C16 alkane + |
| | ethanediamine | branched or linear C14-C18 fatty |
| | | alcohol + branched or linear |
| | | monohydric C4-C5 alcohol |
| 18 | N1,N1-Bis[3-(triethoxysilyl)propyl]-1,2- | branched or linear C14-C16 alkane + |
| | ethanediamine | branched or linear C14-C18 fatty |
| | | alcohol + branched or linear |
| | | monohydric C4-C5 alcohol |
| 19 | N,N-Bis[3-(trimethoxysilyl)propyl]-2-propen-1-amine | branched or linear C14-C16 alkane + |
| | | branched or linear C14-C18 fatty |
| | | alcohol + branched or linear |
| | | monohydric C4-C5 alcohol |
| 20 | N,N-Bis[3-(triethoxysilyl)propyl]-2-propen-1-amine | branched or linear C14-C16 alkane + |
| | | branched or linear C14-C18 fatty |
| | | alcohol + branched or linear |
| | | monohydric C4-C5 alcohol |
| 21 | Methyltrimethoxysilane | branched or linear C14-C16 alkane + |
| | | branched or linear C14-C18 fatty |
| | | alcohol + branched or linear |
| | | monohydric C4-C5 alcohol |
| 22 | Methyltriethoxysilane | branched or linear C14-C16 alkane + |
| | | branched or linear C14-C18 fatty |
| | | alcohol + branched or linear |
| | | monohydric C4-C5 alcohol |
| 23 | Ethyltrimethoxysilane | branched or linear C14-C16 alkane + |
| | | branched or linear C14-C18 fatty |
| | | alcohol + branched or linear |
| | | monohydric C4-C5 alcohol |
| 24 | Ethyltriethoxysilane | branched or linear C14-C16 alkane + |
| | | branched or linear C14-C18 fatty |
| | | alcohol + branched or linear |
| | | monohydric C4-C5 alcohol |
| 25 | Propyltrimethoxysilane | branched or linear C14-C16 alkane + |
| | | branched or linear C14-C18 fatty |
| | | alcohol + branched or linear |
| | | monohydric C4-C5 alcohol |
| 26 | Propyltriethoxysilane | branched or linear C14-C16 alkane + |
| | | branched or linear C14-C18 fatty |
| | | alcohol + branched or linear |
| | | monohydric C4-C5 alcohol |
| 27 | Hexyltrimethoxysilane | branched or linear C14-C16 alkane + |
| | | branched or linear C14-C18 fatty |
| | | alcohol + branched or linear |
| | | monohydric C4-C5 alcohol |
| 28 | Hexyltriethoxysilane | branched or linear C14-C16 alkane + |
| | | branched or linear C14-C18 fatty |
| | | alcohol + branched or linear |
| | | monohydric C4-C5 alcohol |
| 29 | Octyltrimethoxysilane | branched or linear C14-C16 alkane + |
| | | branched or linear C14-C18 fatty |
| | | alcohol + branched or linear |
| | | monohydric C4-C5 alcohol |
| 30 | Octyltriethoxysilane | branched or linear C14-C16 alkane + |
| | | branched or linear C14-C18 fatty |
| | | alcohol + branched or linear |
| | | monohydric C4-C5 alcohol |
| 31 | Dodecyltrimethoxysilane | branched or linear C14-C16 alkane + |
| | | branched or linear C14-C18 fatty |
| | | alcohol + branched or linear |
| | | monohydric C4-C5 alcohol |
| 32 | Dodecyltriethoxysilane. | branched or linear C14-C16 alkane + |
| | | branched or linear C14-C18 fatty |
| | | alcohol + branched or linear |
| | | monohydric C4-C5 alcohol |
| 33 | Octadecyltrimethoxysilane | branched or linear C14-C16 alkane + |
| | | branched or linear C14-C18 fatty |
| | | alcohol + branched or linear |
| | | monohydric C4-C5 alcohol |
| 34 | Octadecyltriethoxysilane | branched or linear C14-C16 alkane + |
| | | branched or linear C14-C18 fatty |
| | | alcohol + branched or linear |
| | | monohydric C4-C5 alcohol |
| 35 | (3-Aminopropyl)triethoxysilan + | branched or linear C14-C16 alkane + |
| | Methyltrimethoxysilan | branched or linear C14-C18 fatty |
| | | alcohol + branched or linear |
| | | monohydric C4-C5 alcohol |
| 36 | (3-Aminopropyl)triethoxysilane + | branched or linear C14-C16 alkane + |
| | Methyltriethoxysilane | branched or linear C14-C18 fatty |
| | | alcohol + branched or linear |
| | | monohydric C4-C5 alcohol |
| 37 | (3-Aminopropyl)triethoxysilane + | branched or linear C14-C16 alkane + |
| | Ethyltrimethoxysilane | branched or linear C14-C18 fatty |
| | | alcohol + branched or linear |
| | | monohydric C4-C5 alcohol |
| 38 | (3-Aminopropyl)triethoxysilane + | branched or linear C14-C16 alkane + |
| | Ethyltriethoxysilane | branched or linear C14-C18 fatty |
| | | alcohol + branched or linear |
| | | monohydric C4-C5 alcohol |
| 39 | (3-Aminopropyl)triethoxysilane + | branched or linear C14-C16 alkane + |
| | Propyltrimethoxysilane | branched or linear C14-C18 fatty |
| | | alcohol + branched or linear |
| | | monohydric C4-C5 alcohol |
| 40 | (3-Aminopropyl)triethoxysilane + | branched or linear C14-C16 alkane + |
| | Propyltriethoxysilane | branched or linear C14-C18 fatty |
| | | alcohol + branched or linear |
| | | monohydric C4-C5 alcohol |
| 41 | (3-Aminopropyl)triethoxysilane + | branched or linear C14-C16 alkane + |
| | Hexyltrimethoxysilane | branched or linear C14-C18 fatty |
| | | alcohol + branched or linear |
| | | monohydric C4-C5 alcohol |
| 42 | (3-Aminopropyl)triethoxysilane + | branched or linear C14-C16 alkane + |
| | Hexyltriethoxysilane | branched or linear C14-C18 fatty |
| | | alcohol + branched or linear |
| | | monohydric C4-C5 alcohol |
| 43 | (3-Aminopropyl)triethoxysilane + | branched or linear C14-C16 alkane + |
| | Octyltrimethoxysilane | branched or linear C14-C18 fatty |
| | | alcohol + branched or linear |
| | | monohydric C4-C5 alcohol |
| 44 | (3-Aminopropyl)triethoxysilane + | branched or linear C14-C16 alkane + |
| | Octyltriethoxysilane | branched or linear C14-C18 fatty |
| | | alcohol + branched or linear |
| | | monohydric C4-C5 alcohol |
| 45 | (3-Aminopropyl)triethoxysilane + | branched or linear C14-C16 alkane + |
| | Dodecyltrimethoxysilane | branched or linear C14-C18 fatty |
| | | alcohol + branched or linear |
| | | monohydric C4-C5 alcohol |
| 46 | (3-Aminopropyl)triethoxysilane + | branched or linear C14-C16 alkane + |
| | Dodecyltriethoxysilane | branched or linear C14-C18 fatty |
| | | alcohol + branched or linear |
| | | monohydric C4-C5 alcohol |
| 47 | (3-aminopropyl)triethoxysilane + | branched or linear C14-C16 alkane + |
| | Octadecyltrimethoxysilane | branched or linear C14-C18 fatty |
| | | alcohol + branched or linear |
| | | monohydric C4-C5 alcohol |
| 48 | (3-aminopropyl)triethoxysilane + | branched or linear C14-C16 alkane + |
| | Octadecyltriethoxysilane | branched or linear C14-C18 fatty |
| | | alcohol + branched or linear |
| | | monohydric C4-C5 alcohol |

A further subject of the present application is the use of the anhydrous carrier medium as contemplated herein for increasing the storage stability of organic silicon compounds and/or for the preparation of an agent for cleaning a keratinous material, an agent for caring for a keratinous material, an agent for caring for and cleaning a keratinous material, an agent for coloring a keratinous material and/or an agent for temporarily reshaping a keratinous material.

With regard to further preferred embodiments of use, what has been said about the anhydrous carrier media applies mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An anhydrous carrier medium for the storage stabilization of organic silicon compounds, the anhydrous carrier medium comprising:
a) at least one organic silicon compound,
b) at least one branched or linear C8-C30 alkane,
c) at least one branched or linear C10-C30 fatty alcohol, and
d) at least one branched or linear C2-C8 monohydric alcohol.

2. The anhydrous carrier medium according to claim 1,
wherein
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R₅, R_{5′}, R_{5″}, R₆, R_{6′} and R_{6″} stand independently for a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
(A″″)—Si(R₆″)_{d″}(OR₅″)_{c″} (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
wherein at least one of the components of e, f, g and h is different from 0.

3. The anhydrous carrier medium according to claim 1,
wherein
the anhydrous carrier medium comprises at least one organic silicon compound of the formula (II) which is selected from the group of
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-Methyl-3-(triethoxysilyl)-N-[3-(triethoxysilyl)propyl]-1-propanamine,
2-[Bis[3-(trimethoxysilyl)propyl]amino]-ethanol,
2-[Bis[3-(triethoxysilyl)propyl]amino]ethanol,
3-(Trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-propen-1-amine, and
N,N-Bis[3-(triethoxysilyl)propyl]-2-propen-1-amine.

4. The anhydrous carrier medium according to claim 1, wherein the anhydrous carrier medium comprises at least one organic silicon compound of formula (IV),
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

5. The anhydrous carrier medium according to claim 1, wherein the anhydrous carrier medium comprises at least two structurally different organic silicon compounds.

6. The anhydrous carrier medium according to claim 1, wherein the organic silicon compound is present in the anhydrous carrier medium in an amount of from about 0.01 to about 10% by weight.

7. The anhydrous carrier medium according to claim 1, wherein the alkane is a C10-C24 alkane and/or wherein the alkane is present in the anhydrous carrier medium in an amount of from about 1 to about 50% by weight, and/or wherein
the fatty alcohol is a C12-C24 fatty alcohol, and/or the fatty alcohol is present in the anhydrous carrier medium in an amount of from about 5 to about 50% by weight, and/or wherein
the alcohol is a C3-C6 alcohol, and/or wherein the alcohol is present in the anhydrous carrier medium in an amount of from about 4 to about 50% by weight.

8. A cosmetic composition comprising an anhydrous carrier medium according to claim 1 and an aqueous phase, wherein the anhydrous carrier medium or the aqueous phase comprises a cationic surfactant having one or more C8-C22 alkyl groups, or wherein the cationic surfactant is one of the following formula, 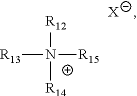
wherein
R₁₂, R₁₃, R₁₄ independently represent a C1-C6 alkyl group, a C2-C6 alkenyl group or a C2-C6 hydroxyalkyl group,
R₁₅ represents a C8-C28 alkyl group, and
X— stands for a physiologically compatible anion,
or wherein the cationic surfactant is one of the following formula, 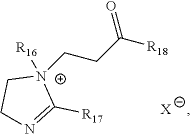
wherein
R₁₆ stands for a C1-C6 alkyl group,
R₁₇, R₁₈ are independently a C7-C27 alkyl group, and
X— stands for a physiologically compatible anion,
or wherein the cationic surfactant is one of the following formula, 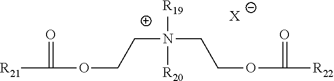
wherein
R₁₉, R₂₀ independently represent a C1-C6 alkyl group, a C2-C6 alkenyl group or a C2-C6 hydroxyalkyl group,
R₂₁, R₂₂ are independently a C7-C27 alkyl group, and
X— stands for a physiologically compatible anion,
or wherein the cationic surfactant is one of the following formula,
NR₂₃R₂₄R₂₅,
wherein
R₂₃, R₂₄ independently represent a C1-C6 alkyl group, a C2-C6 alkenyl group or a C2-C6 hydroxyalkyl group, and
R₂₅ represents a C8-C28 alkyl group, and/or
wherein the cationic surfactant is present in the aqueous phase in an amount of from about 0.1 to about 5% by weight.

9. The cosmetic composition according to claim 8, wherein the anhydrous carrier medium or the aqueous phase comprises a nonionic surfactant, wherein the nonionic surfactant is a fatty alcohol ethoxylate in which the fatty alcohol portion of the fatty alcohol ethoxylate has an alkyl chain length of C4-C30, and/or in which the number of ethoxy groups in the fatty alcohol ethoxylate is from about 2 to about 120, and/or wherein the nonionic surfactant is present in an amount of from about 0.1 to about 10% by weight.

10. The cosmetic composition according to claim 8, wherein the anhydrous carrier medium or the aqueous phase comprises an anionic surfactant, wherein the anionic surfactant is preferably a fatty alcohol sulfate with a chain length of C8-C22, and/or wherein the anionic surfactant is present in the aqueous phase in an amount of from about 0.1 to about 10% by weight.

11. The cosmetic composition according to claim 8, wherein the weight ratio of the anhydrous carrier medium to the aqueous phase is from about 1 to 10 to about 10 to 1.

12. A method comprising using an anhydrous carrier medium according to claim 1
to increase the storage stability of organic silicon compounds,
for the preparation of an agent for cleaning a keratinous material, an agent for caring for a keratinous material, an agent for caring for and cleaning a keratinous material, an agent for coloring a keratinous material and/or an agent for temporarily reshaping a keratinous material.

13. The anhydrous carrier medium according to claim 4, wherein the at least one organic silicon compound of formula (IV) is selected from the group of:
Methyltrimethoxysilane,
Methyltriethoxysilane,
Ethyltrimethoxysilane,
Ethyltriethoxysilane,
Propyltrimethoxysilane,
Propyltriethoxysilane,
Hexyltrimethoxysilane,
Hexyltriethoxysilane,
Octyltrimethoxysilane,
Octyltriethoxysilane,
Dodecyltrimethoxysilane,
Dodecyltriethoxysilane,
Octadecyltrimethoxysilane, and
Octadecyltriethoxysilane.

14. The anhydrous carrier medium according to claim 1, wherein the alkane is a C12-C18 alkane.

15. The anhydrous carrier medium according to claim 1, wherein the alkane is a C14-C16 alkane.

16. The anhydrous carrier medium according to claim 1, wherein the alkane is present in the anhydrous carrier medium in an amount of from about 1 to about 50% by weight.

17. The anhydrous carrier medium according to claim 1, wherein the fatty alcohol is a C12-C24 fatty alcohol.

18. The anhydrous carrier medium according to claim 1, wherein the fatty alcohol is a C14-C18 fatty alcohol.

19. The anhydrous carrier medium according to claim 1, wherein the fatty alcohol is present in the anhydrous carrier medium in an amount of from about 5 to about 50% by weight.

20. The anhydrous carrier medium according to claim 1, wherein the alcohol is a C3-C6 alcohol.
